(19) United States
(12) Patent Application Publication
(10) Pub. No.: US 2006/0171998 A1
(43) Pub. Date: Aug. 3, 2006

(54) Sanitary Wipes and Seat Covers for gym equipment

(76) Inventor: Burnetta Sherelzae Trimble, Decatur, GA (US)

Correspondence Address: Ms.Burnetta Sherelzae Trimble, 2960 Hampton Place, Decatur, GA, 30034, US

(21) Appl. No.: 10/906,063

(22) Filed: Feb. 1, 2005

(51) Int. Cl. A61K 9/70; A61F 13/00

(52) U.S. Cl. 424/443

(57) ABSTRACT

Wipes: Large in size to clean gym equipment disinfectant and sanitizes equipment disposable Seat Covers: Large and small in size to accommodate gym equipment Protects from perspiration Velcro for easy detaching disposable

The wipes are large in size but are similar to regular wipes. The seat covers are attached to the gym equipment by a Velcro. Same affect as a baby's pamper, the inside soaks the moisture and it keeps your skin dry as well as the equipment. The sweat does not leak through the material. The cover is strong enough to be used from equipment to equipment before disposing.

What is claimed is:

1. The wipes will contain an anti bacterial disinfectant to minimize the possibility of infection from human sweat. Both products will protect consumers from germs and infections that may be caused from human perspiration.
